(19) United States
(12) Patent Application Publication
(10) Pub. No.: US 2025/0032980 A1
(43) Pub. Date: Jan. 30, 2025

(54) Method for Sequestering Carbon

(71) Applicant: Jan-Heiner Küpper, Kleinkoschen (DE)

(72) Inventors: Jan-Heiner Küpper, Kleinkoschen (DE); Tassilo Küpper, Senftenberg (DE); Friedrich Jung, Dresden (DE); Conrad Jung, Dresden (DE)

(21) Appl. No.: 18/717,715

(22) PCT Filed: Dec. 8, 2022
(86) PCT No.: PCT/EP2022/084959

§ 371 Date: Jun. 7, 2024

(30) Foreign Application Priority Data
Dec. 8, 2021 (DE) 102021214010.6

(51) Int. Cl. B01D 53/84; B01D 53/62; C12N 1/12

(52) U.S. Cl. CPC B01D 53/84; B01D 53/62; C12N 1/12; B01D 2251/95; B01D 2257/504; B01D 2258/06

(57) ABSTRACT

The object of the invention is a process for sequestering carbon or removing carbon dioxide from air using a bioreactor equipped with autotrophic microorganisms, where the carbon dioxide from the air is the only carbon source.

The object of the invention is a process for sequestering carbon or removing carbon dioxide from air using a bioreactor equipped with autotrophic microorganisms, wherein the carbon dioxide from the air is the sole carbon source.

The climate targets adopted at the COP21 conference in Paris in 2015 set the UN member states the task of limiting the global average temperature increase to well below 2° C., and preferably no higher than 1.5° C. An internationally acclaimed publication highlights the danger that if these so-called “Paris climate targets” are not met, the planet could enter a “Hothouse Earth” phase for an unforeseeably long time due to self-reinforcing processes—with probably serious consequences for human society and ecosystems (Steffen et al., 2018. Trajectories of the Earth System in the Anthropocene. www.pnas.org/cgi/doi/10.1073/pnas.1810141115). However, most UN member states are repeatedly lagging behind even their own nationally proposed climate targets: since the Paris Climate Agreement, global CO2 emissions have risen continuously until 2019 (http://www.globalcarbonatlas.org/en/CO2-emissions). Caused by the Covid-19 pandemic and the resulting global economic upheaval, global CO₂ emissions fell by up to 5.8% in 2020, the biggest drop since the Second World War (https://www.iea.org/articles/global-energy-review-co2-emissions-in-2020). With the onset of economic recovery, CO₂ emissions also rose again in 2021. With the emission reduction targets proposed by the countries to date, the earth is currently on a path that makes a critical temperature increase of 2.4° C. likely towards the end of the century—if the reduction targets are not met, there is even a threat of around 3° C. (https://climateactiontracker.org/press/global-update-projected-warming-from-paris-pledges-drops-to-two-point-four-degrees/).

Climate research experts have identified the following three general measures that need to be taken today and in the future, both individually and in combination, in order to stabilize climate systems in the long term and avoid the Hothouse Earth scenario:

Most important is the dramatic reduction in emissions of anthropogenic greenhouse gases (especially CO₂, but also methane and nitrous oxide from agriculture), driven primarily by a significant CO₂ price (including CO₂ equivalents) and an increasing supply of CO₂-neutral technologies.

The third general measure is the development of efficient, economically viable and ecologically compatible technologies for carbon sequestration from the atmosphere, also known as CO₂ sequestration. Experts are familiar with the Bioenergy with Carbon Capture and Storage (BECCS) strategy, among others. BECCS is seen by the Intergovernmental Panel on Climate Change (IPCC) as a way of fixing large quantities of atmospheric CO₂ via plant growth (Climate Change 2014, Mitigation of Climate Change, Working Group III Contribution to the Fifth Assessment Report of the Intergovernmental Panel on Climate Change). The fixation of CO₂ via photosynthesis enables plant growth. The plants are then fermented in a biotechnological process known to experts to produce biogas, which is used, for example, in a gas-fired power plant to generate electricity. The CO₂ released from the biogas through combustion is captured (CO₂ Capture) and stored in the ground (CO₂ Storage). However, experts are critical of the potential of BECCS to remove CO2 from the atmosphere because the cultivation of plants for BECCS would compete with agricultural use and thus with food production. Williamson concludes that the Earth's usable land areas are far from sufficient to sequester enough CO₂ from the atmosphere by 2100 via BECCS, and that additional CO₂ emissions will even be generated if forests are converted into cropland for BECCS (Williamson, P. Emissions reduction: Scrutinize CO₂ removal methods. Nature 530, pages 153-155, 2016. https://doi.org/10.1038/530153a). Experts are aware of other methods for carbon sequestration such as air capture via CO₂ filter systems with subsequent storage of the CO₂. This strategy is currently still associated with high energy and cost expenditure. Storing large quantities of CO₂ in the ground can also pose a considerable risk in the event of earthquakes, for example.

Overall, technical measures for carbon sequestration—also known as negative CO₂ emissions—are absolutely essential if measures to reduce greenhouse gases or reforestation projects are not sufficient. This combination can still pave the way for a “Stabilized Earth” instead of a “Hothouse Earth”. The measures must take effect by 2050 at the latest.

One promising option for carbon sequestration is the use of bioreactors that contain autotrophically growing microorganisms. Mostly algae, microalgae and cyanobacteria are used, which extract CO₂ from the atmosphere and form biomass from it. This biomass can be put to various uses such as 1) biogas production for energy production, 2) extraction of carbon compounds for the chemical industry, 3) biofuels, 4) food additives, which are mainly contained in microalgae, 5) other valuable substances such as pharmaceutical substances and cosmetics. Carbon sequestration occurs when carbon compounds with a long life cycle are produced from the biomass of microalgae, e.g. carbon fibers, which are used as building or construction materials. Techniques are known to the skilled person by which chemically stable carbon compounds can be generated from the biomass of microalgae. In the long term, the biomass from microalgae could also be deposited in disused mines in such a way that no CO₂ or methane can escape. Overall, the skilled person is aware of several strategies and processes for keeping the carbon bound in the biomass of microalgae sequestered in the long term and preventing it from being released back into the atmosphere as CO₂ or methane.

Interestingly, eukaryotic microalgae such as Chlorella but also cyanobacteria such as Arthrospira have a high CO2 fixation efficiency per area and year compared to land plants. The sequestration of CO₂ in microalgae cultivation systems has a number of advantages, such as a light use efficiency 10-50 times greater than that of land plants, high growth rates and consequently high CO₂ biofixation rates (Cheng J, Huang Y, Feng J, Sun J, Zhou J, Cen K. Improving CO2 fixation efficiency by optimizing Chlorella PY-ZU1 culture conditions in sequential bioreactors. Bioresour Technol. 2013; 144:321-327). Furthermore, it is known to the skilled person that a variety of high-value products can be obtained from cyanobacteria, such as food for humans and animals that can be used in the food sector, biologically active compounds, nutraceuticals and even in the energy sector using biomass for biofuels.

In this sense, biological CO₂ fixation with microalgae is considered to be very effective (Morais M G, Costa J A V. J Biotechnol. 2007; 129:439-445; Andrea C T C et al, Computational Vision and Bio-Inspired Computing. 2019; 697-707). Microalgae are able to convert inorganic carbon from the atmosphere into organic carbon biomass. Like green plants, cyanobacteria exhibit high oxygen-emitting photosynthetic efficiency and possess a number of microbial traits, including a rather fast growth rate, a high content of valuable proteins, and a variable metabolism that responds rapidly to environmental changes. The photosynthesis of microalgae offers the possibility of fixing CO₂ using sunlight, converting the carbon into biomass and at the same time releasing oxygen. Microalgae contain around 50% (w w−1) carbon in their biomass. During photosynthesis, 1 mole of CO₂ and 1 mole of water are used to produce 1 mole of organic carbon (CH₂ O) and 1 mole of O. ₂

Furthermore, the products obtained directly are all advantageously CO₂-neutral in production.

In the state of the art, WO2019086656A1, WO 1998/045409A1 and EP 2 568 038 A1 describe laminar photobioreactors for the production of autotrophic microorganisms, in particular microalgae, whereby the following technical problems are solved:

a.) A continuous CO₂ supply is ensured, as the atmospheric CO₂ concentration of 400 ppm (0.04%) does not allow optimal growth of microalgae, for example. At optimum CO₂ concentrations, it has been found that microalgae are about 10-50 times more efficient in the formation of biomass than land plants. The technical theory describes that microalgae such as Chlorella, Scenedesmus, Spirulina, Nannochloropsis, Nostoc and Chlorococcus can grow very well in the range of 1-20% CO₂ (i.e. approx. 25 to 500 times higher than in the atmosphere) and have a correspondingly high biomass productivity (see also Appl. Biochem Biotechnology, 2016 179:1248-1261 and literature cited therein). So far, however, chemically pure CO₂ (technical CO₂) has been used. This does not solve the problem of carbon sequestration, as this CO₂ is obtained as a chemical product in a very energy-intensive process. Various working groups have already tried to use alternatives in the form of exhaust gas streams from power plants. Although this would allow the CO₂ produced during the combustion of fossil fuels to be sequestered, it would not guarantee the direct removal of CO₂ from the atmosphere. It is also known that exhaust gas streams from power plants contain impurities such as sulphur, nitrogen oxides, carbon monoxide and heavy metals, which can strongly inhibit the growth of microorganisms and contaminate products. The removal of harmful impurities from these exhaust gas streams is very cost-intensive.

b.) Bioreactors, especially photobioreactors, can maintain the optimal growth conditions of the microorganism, such as temperature, pH value, nutrients, etc.

c.) WO2019086656A1 describes a suitable device of a bioreactor including a scraper for capturing carbon dioxide from the air, so-called Air Capture Module (carbon dioxide extraction plant of the company Climeworks in Switzerland (http://www.climeworks.com)), whereby atmospheric carbon dioxide is bound with the aid of an adsorber material and, after treatment with heat or vacuum, the atmospheric carbon dioxide is stored in a pressurized container.

However, the prior art does not describe a process for sequestering carbon from the air using a bioreactor equipped with autotrophic microorganisms, where the carbon dioxide from the air is the only carbon source.

The invention therefore relates to the task of providing a process for sequestering carbon from the air by producing biomass from autotrophic microorganisms in a bioreactor.

A further object of the invention relates to the task of avoiding phototoxicity or photoinhibition by a process according to the invention.

Therefore, to solve this first problem, the invention relates to a process for sequestering carbon from the air using a bioreactor equipped with autotrophic microorganisms, wherein the carbon dioxide from the air is the sole carbon source.

For the purposes of the present invention, the term “autotrophic microorganisms” comprises microorganisms which utilize light as an energy source (photoautotrophic microorganisms) or a chemical energy source (e.g. hydrogen) (chemoautotrophic microorganisms). Autotrophic microorganisms are able to fix carbon dioxide and in this way produce biomass so that carbon dioxide is removed from the environment.

For the purposes of the present invention, algae are understood to be photosynthetic organisms that live in water and have a cell nucleus. For the purposes of the present invention, microalgae are understood to mean microscopically small and generally unicellular algae. In this respect, cyanobacteria (in particular blue-green algae) are also counted as microalgae, although these are prokaryotic (non-nucleated) organisms which belong to the group of bacteria.

In the context of the present invention, autotrophic microorganisms are understood to mean photoautotrophic microorganisms or chemoautotrophic microorganisms, in particular such as archaebacteria, algae, microalgae, cyanobacteria, in particular algae of the genera Chlorella, Scenedesmus, Arthrospira, Nannochloropsis, Nostoc or Chlorococcus.

However, a preferred embodiment is the genus Arthrospira.

The term “Arthrospira” as used in the present invention refers to a genus of free-swimming, filamentous, multicellular, photosynthesizing cyanobacteria characterized by cylindrical, multicellular trichomes in an open left-hand helix. A dietary supplement is preferably prepared from the biomass of A. platensis and/or A. maxima, known as spirulina. Spirulina is a common and commercial name for a variety of similar cyanobacteria species belonging to the genus Arthrospira. The genus Arthrospira includes, but is not limited to, the following species A. platensis, A. maxima, A. fusiformis, A. indica, A. innermongoliensis, A. jenneri, A. massartii and A. erdosensis.

According to the present invention, the following species are preferred, namely A. platensis, A. maxima and A. fusiformis, since a high productivity for the purpose of sequestering CO₂ from the air can be achieved according to the invention.

For the purposes of the present invention, a “bioreactor” is a facility for the production of microorganisms outside their natural environment and within an artificial technical environment. In a photobioreactor, photosynthetic microorganisms or microalgae are cultivated. A natural or artificial light source can be used. A bioreactor can be a closed system, e.g. a glass tube photobioreactor, or an open system, e.g. an open-pond bioreactor. The latter can be located in the open air or under a glass or foil roof. A photobioreactor or a device as described in WO2019086656A1, WO 1998/045409A1 and EP 2 568 038 A1 is also preferred. Such a bioreactor can be equipped with autotrophic microorganisms.

“Air” in the sense of the present invention is natural, atmospheric air, usually containing 78% by volume nitrogen, 21% by volume oxygen and noble and other gases, in particular carbon dioxide, also of anthropogenic origin, also known as atmospheric carbon dioxide.

Arthrospira in particular uses atmospheric CO₂ indirectly for photosynthesis via hydrogen carbonate (HCO₃⁻), so-called bicarbonate or carbonate (CO₃²⁻). In the aqueous culture medium, there is a pH-dependent equilibrium between CO₂ and H₂ CO₃ (carbonic acid), which can have a pH value of 7 to 11, whereby the primary carbon source HCO₃⁻ for Arthrospira enters the cell by active transport (Cogne G, et al., Biotechnol Bioeng. 2003; 84:667-676).

In the state of the art, a liquid-aqueous growth or culture medium with sodium hydrogen carbonate as carbon source (so-called Zarrouk medium according to example 4) is used in the cultivation of Arthrospira (Zarrouk C. Contribution à l'étude d'une cyanophycée. Influence de divers facteurs physiques et chimiques sur la croissance et photosynthèse de Spirulina maxima, Ph.D. thesis, University of Paris, 1966).

Usually, such Arthrospira cultures are aerated with an air-gas flow so that the oxygen formed can be removed. The air flow can contain the atmospheric CO₂ concentration of 0.041% (410 ppm), but in some systems the CO₂ concentration is increased even further. This is to firstly either maintain/lower the pH value and/or secondly to act as an additional inorganic carbon source. As a result, however, the additionally added CO₂ escapes from the bioreactor unused, as only a negligible proportion of the CO₂ is converted into HCO₃⁻ ions. The HCO₃⁻ of the medium originating from the NaHCO₃ can also be released via the hydrogen carbonate/CO₂ equilibrium (carbonic acid equilibrium) if no equilibrium has yet been established. This can even lead to the fact that, if very high hydrogen carbonate concentrations are normally used, no atmospheric CO₂ is fixed in the balance, but on the contrary CO₂ is even released into the atmosphere.

According to the invention, however, a culture medium for autotrophic microorganisms is used which has no carbon source. In particular, Arthrospira can be cultivated in a medium without the addition of a carbon source, in particular hydrogen carbonate (see example 4). The only carbon source is provided in the form of atmospheric air or air flow with 0.041% CO₂ In a further preferred embodiment, the photon flux density is taken into account for the method according to the invention.

The photosynthetic apparatus of Arthrospira is organized in layers of lipoprotein membranes (thylakoids) and aqueous phases, the cytosol. Oxygen-emitting photosynthesis drives biosynthesis at the expense of inorganic nutrients. It is generally observed that the higher the intensity of the light source (photon flux density, PFD for short), the stronger the growth of Arthrospira. The absolute highest biomass concentrations and productivity can be achieved at the highest PFD if there are no other limitations due to nutrients (C, N, P, S) or abiotic factors such as temperature or pH (Jung F, Jung C G H, Krüger-Genge, Waldeck P, Kupper J H. Factors influencing the growth of Spirulina platensis in closed photobioreactors under CO₂— O₂ conversion. J Cellular Biotechnology. 2019; 5:125-134).

However, under the limiting conditions of the lack of a carbon source in the culture medium, too strong a PFD can have two detrimental effects:
i.) photoinhibition, which manifests itself in a microalgae culture as a reduction in the rate of photosynthesis, and
ii.) Photooxidation, which has lethal effects on the cells and can lead to total loss of the culture (Jensen S, Knutsen G. Influence of light and temperature on photoinhibition of photosynthesis in Spirulina platensis. Journal of Applied Phycology. 1993; 5:495-504), see FIGS. 1 and 2.

The inventors have found that the cultivation of Arthrospira with an inappropriately high PFD, especially at the beginning of cultivation, can lead to a toxic light effect and even complete death of the cultures due to the excess of light energy equivalents.

This occurs in particular if the inoculum comes from a backup reactor that is in the stationary phase. This is probably due to the fact that the amount of phycocyanin is increased over time in cultures that are in the stationary phase. If these cultures are diluted and the PFD applied is too high, the light-harvesting complexes or the electron transport chain are saturated or overloaded. A possible explanation could be that the activity of RuBisCO (ribulose-1,5-bisphosphate carboxylase/oxygenase) is too low to provide sufficient carbon scaffolds for the synthesis of substances. This ultimately leads to the formation of oxygen radicals, which are responsible for phototoxicity.

This effect no longer occurs just a few days after inoculation. At this point, Arthrospira cells have already adapted to the light conditions. This occurs through a reduction in the light-collecting complexes and possibly through an increase in the amount of carotenoids. According to the inventors, Arthrospira is equipped with a considerable and relatively rapid adaptability in this respect.

Therefore, the invention relates to a further embodiment, namely a method for preventing or avoiding phototoxicity or photoinhibition with the aid of light and/or air supply, in particular the control thereof.

In addition, this procedure according to the invention allows HCO₃⁻ to be advantageously provided to the autotrophic microorganisms, in particular microalgae such as Arthrospira, in steady-state, and consequently continuous growth is possible.

The CO₂ in the air stream is converted to CO in the water in the pH range relevant for Arthrospira (see Hägg diagram, see http://www.wissenschaft-technik-ethik.de/wasser_ph.html#330, FIG. 9) by consuming a water molecule:

CO₂+H₂O→H⁺+HCO₃⁻.

The HCO₃⁻ produced is consumed by Arthrospira to build up biomass, whereby an H⁺ ion is also taken up from the aqueous medium at the same time. The more light, the higher the HCO₃⁻ consumption. HCO₃⁻ is replicated by CO₂ in the air-gas flow, i.e. a large air-gas flow leads to a higher formation rate, as does an increased CO₂ concentration. To avoid photooxidation or inhibition, the HCO₃⁻ formation rate should on average be greater than or equal to the HCO₃⁻ consumption rate by the Arthrospira cells, in the sense of a steady state. The inventors have observed an increase in pH whenever the HCO₃⁻/CO₂ equilibrium is first established in the Arthrospira nutrient solution, followed by exposure to light, which enables photosynthesis and consequently autotrophic growth. An increase in the pH value therefore indicates that more HCO₃⁻ is being consumed than is being produced. A drop in the pH value means that more HCO₃⁻ is being formed than is being consumed.

In order to ensure a sufficient HCO₃⁻ formation rate, CO₂ must be added to the culture medium. This can preferably be taken from the circulating air (with 0.041% CO₂) or, if necessary, concentrated technical CO₂ is added to the circulating air and consequently the total CO₂ concentration in the culture medium is increased. The air flow can preferably be pumped into the bioreactors via a compressor.

These observations on photoinhibition give rise to the following problem, which is solved in full in the context of the invention:
1. To utilize the potential of fast-growing autotrophic microorganisms, especially microalgae such as Arthrospira, for atmospheric carbon sequestration, the microalgae must grow without a carbon source such as sodium hydrogen carbonate (NaHCO₃) added to the culture medium, as otherwise the cultures not only do not absorb atmospheric CO₂ but the cultures can even become a source of CO₂ emissions by shifting the HCO₃⁻/CO₂ equilibrium.
2. Since PFD exposure, which is common for closed bioreactors as well as open pond systems, can lead to the observed photoinhibition/oxidation, especially in hydrogen carbonate-free Arthrospira cultures, adapted cultivation conditions should be found that allow the growth of Arthrospira cultures despite the lack of hydrogen carbonate.
3. Due to the constantly changing conditions (light intensity, temperature, CO₂ concentration, etc.), especially in open pond systems in the open air, it is necessary to dynamically adjust the HCO₃⁻ formation/consumption rate to the existing cultivation conditions.

In accordance with the invention, the inventors have cultivated Arthrospira exclusively with atmospheric CO₂ from the air and without added sodium hydrogen carbonate, without photoinhibition/oxidation of the Arthrospira cultures occurring.

For the first time, it is possible to use autotrophic microorganisms, especially microalgae such as Arthrospira, for atmospheric carbon sequestration and consequently generate negative CO emissions. ₂

The solution to the problem consists of the following steps according to the invention:

In particular, Arthrospira cultures without hydrogen carbonate supplementation are exposed to a PFD of preferably greater than 0-10,000 μE/m²*sec, in particular 15-2,000 μE/m²*sec, preferably at a temperature between 15 and 45° C. and a pH value between 7 and 11, for a period of preferably 5-30 days after inoculation until the first harvest. In principle, there are various control options, as the PFD is linked both to the applied air flow, preferably from 50-50,000 L/h*m² in particular 100-2,000 L/h*m², and to the CO₂ concentration in the culture medium. In all three variants listed, the control according to the invention is based on using the pH value as a control variable so that the hydrogen carbonate concentration in the culture medium does not fall below a critical value and consequently photooxidation is effectively prevented. This can be controlled as an additional test variable via the oxygen concentration in the medium.
1. The automatic control of the growth process of Arthrospira in the bioreactor is achieved by two mechanisms:
   i.) by controlling the pH value via the PFD by a defined pH setpoint value between 7 and 11, and
   ii.) by regularly adjusting the pH setpoint to the actual conditions. In the bioreactor, the control is carried out by varying the PFD so that the pH value remains constant within a defined fluctuation range. Such a fluctuation range is preferably 0.01-0.1 to 1 pH unit. As the pH value increases, the PFD is reduced from a pH difference of e.g. 0.01 to the target value; if the pH value falls again, the PFD is increased. The specified pH difference of 0.01 is exemplary; a larger or smaller difference is also possible within the scope of the invention. This control mechanism prevents the hydrogen carbonate concentration in the AP nutrient medium from becoming subcritical and photoinhibition/oxidation processes from taking place as a result. The control is carried out with a conventional controller, e.g. with the aid of an 8-bit (256 discrete steps) PID controller. The PFD to be applied is recalculated and adjusted every 10 seconds. FIG. 3 shows the process of a PFD adjustment. The PFD is adapted to each pH change as shown in the figure and in this way keeps the pH setpoint at the same level. The pH setpoint is determined at the start of cultivation and automatically over time (every 2-12 hours). The control of the PFD keeps the pH value in the culture medium constant within a permitted fluctuation range; this is achieved by adjusting the consumption of HCO₃⁻ in the culture medium to the rate of HCO₃-formation. If the HCO₃⁻ consumption by Arthrospira is greater than the HCO₃⁻ formation rate by the air flow through the culture medium, the pH increases according to the observation of the inventors. In this case, the PFD is reduced according to the invention, so that the pH decreases (see FIG. 3). If, on the other hand, the HCO₃⁻ consumption by Arthrospira is lower than the HCO₃-formation rate, the pH decreases. In this case, the PFD is increased, which leads to an increase in the pH value (see FIG. 3).
   As the cultivation conditions and therefore the pH value can change with increasing cultivation time, the pH setpoint can be regularly adjusted to the changed conditions. When the lighting is switched off, photosynthesis by Arthrospira comes to a standstill so that no more HCO₃⁻ is consumed from the culture medium. An equilibrium is then established between the air flow, which supplies 0.04% CO₂, and the dissolved CO₂ in the culture medium (which is then converted into HCO₃⁻ according to the Hägg diagram (supra)). This leads to a lowering of the pH value. However, once equilibrium is reached, the pH cannot decrease further and remains constant. To determine the pH equilibrium value (=target value), the PFD is switched off, the PID controller or control unit is deactivated and the change in the pH value is monitored. As soon as the pH value (dashed line) no longer falls (constant pH values over 10 minutes), this pH value+0.05 is set as the target value (see FIG. 4).
2. In a further embodiment of the invention, the control can be realized in such a way that the volume of the air flow is adapted to the PFD. This is particularly relevant for open bioreactors. The air flow and thus the HCO₃⁻ formation rate is adapted to the PFD present in such a way that photooxidation is prevented. However, the pH value between 7 and 11 is also the control variable here.
3. In another embodiment of the invention, instead of increasing the air flow, the air flow is kept constant and CO₂ preferably obtained from the atmosphere is increased to 0.1%-5%. This can be achieved, for example, by using an air capture module for the reversible binding and concentration of atmospheric CO₂. Air capture modules that can achieve this are known to the skilled person. In this variant, some of the additionally supplied CO₂ may escape from the bioreactor back into the atmosphere without any interaction, which is disadvantageous from an energy point of view. One advantage is that, in purely quantitative terms, more atmospheric CO₂ is converted into biomass.

In a closed photobioreactor, the radiation intensity of the light source can be adaptively adjusted using an algorithm linked to the pH value. In open-pond systems, which are located in a greenhouse, a foil tunnel or completely outdoors, for example, an optimum level of PFD can be ensured using automatically controllable sun sails.

The air flow is preferably pumped into the bioreactors via a compressor.

Therefore, the invention relates to a process for sequestering carbon from air according to one of the above embodiments, wherein the pH of the culture medium is 7 to 11, and is adjusted via photon flux density (PFD) and/or with an air stream and/or with additional carbon dioxide in the air stream.

Furthermore, the invention relates to a method for sequestering carbon from air according to one of the above embodiments, wherein the pH of the culture medium represents a target value between 7 to 11, and deviations from the target value are controlled via the photon flux density (PFD) and/or with an air stream and/or with additional carbon dioxide in the air stream.

In addition, the invention relates to a process for sequestering carbon from air according to one of the above embodiments, wherein the photon flux density (PFD) is greater than 0-10,000 μE/m²*sec, in particular 15-2000 μE/m2*sec and/or the air is fed to autotrophic microorganisms in at least one bioreactor with an air flow of 50-50.000 L/h*m², in particular 100-2000 L/h*m² and/or with additional carbon dioxide in the air flow, whereby the proportion of carbon dioxide in the air flow is up to 0.1-5% by volume.

In a further embodiment, the culture medium is gassed with the aid of tubes which are fed into the culture medium.

In addition, the invention relates to a process for sequestering carbon from air according to one of the above embodiments, wherein the temperature in the bioreactor is between 2° and 45° C.

The process according to the invention therefore allows the prevention or avoidance of phototoxicity or photoinhibition and the provision of HCO₃⁻ in steady-state for the autotrophic microorganisms, in particular microalgae such as Arthrospira.

A further advantage of the invention is that the biomass production of Arthrospira can generate skimming when fumigated with air, whereby the contamination with debris (debris of perishing cells), bacteria or ciliates, flagellates (eukaryotic protozoa) is considerably lower than in conventional propagation in a bioreactor with Zarrouk complete medium including added HCO₃⁻ as a carbon source according to the prior art.

The process according to the invention therefore leads to advantageous skimming, whereby foreign bacteria etc. accumulate in the protein-rich foam and can be easily skimmed off. As a result, a pure product of Arthrospira is obtained.

The CO₂-neutral biomass obtained and produced can be used for the usual applications, such as the production of biofuel, chemical substances, energy utilization, etc. (supra).

The invention further relates to a biomass comprising or consisting of autotrophic microorganisms, in particular Arthrospira obtainable by a process according to the invention, in particular produced according to example 5, wherein the culture medium does not contain a carbon source and the nitrogen content in the biomass is at least 10 wt. %, in particular at least 13 wt. %, or the protein content is more than 70 wt. %, in particular more than 75 wt. %, in particular at least 80 wt. %. In particular, the content of phycocyanin in the biomass obtained is more than 10% by weight, in particular more than 15% by weight. In particular, the content of allophycocyanin in the biomass obtained is more than 3% by weight, in particular more than 4% by weight. In particular, the content of fatty acids in the biomass obtained is more than 5% by weight, in particular more than 2% by weight of palmitic acid. The weight information refers to the dry weight of the biomass obtained.

Furthermore, the invention also relates to a method according to the invention for producing such a biomass.

The following examples and illustrations serve to explain the invention without, however, limiting the object of the invention.

### FIGURES

FIG. 1:

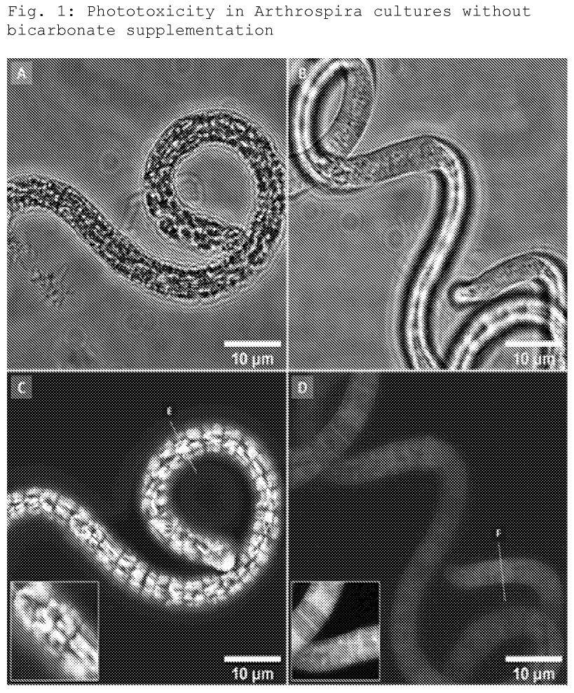

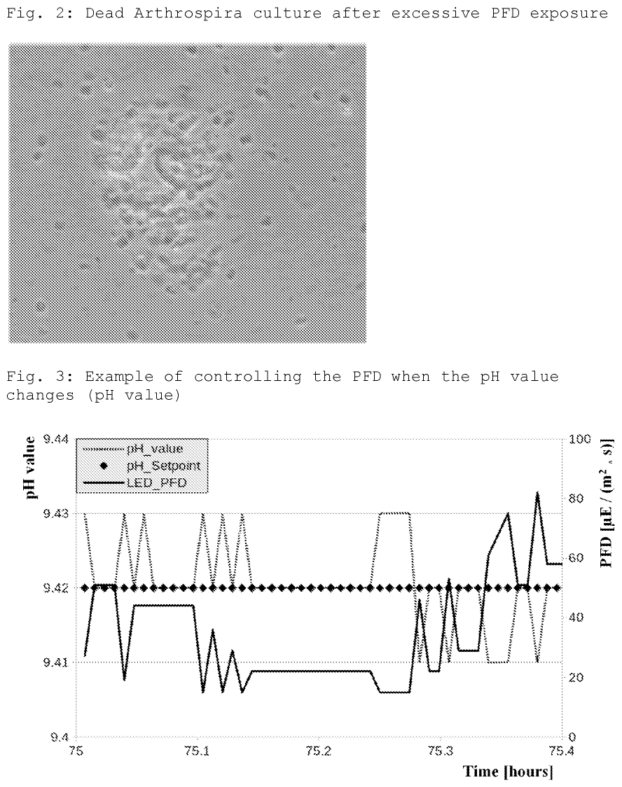

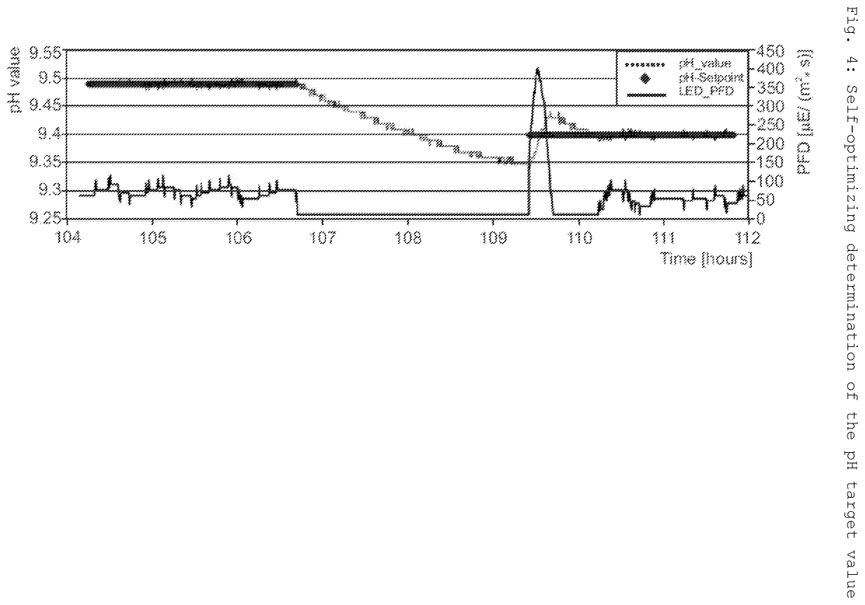

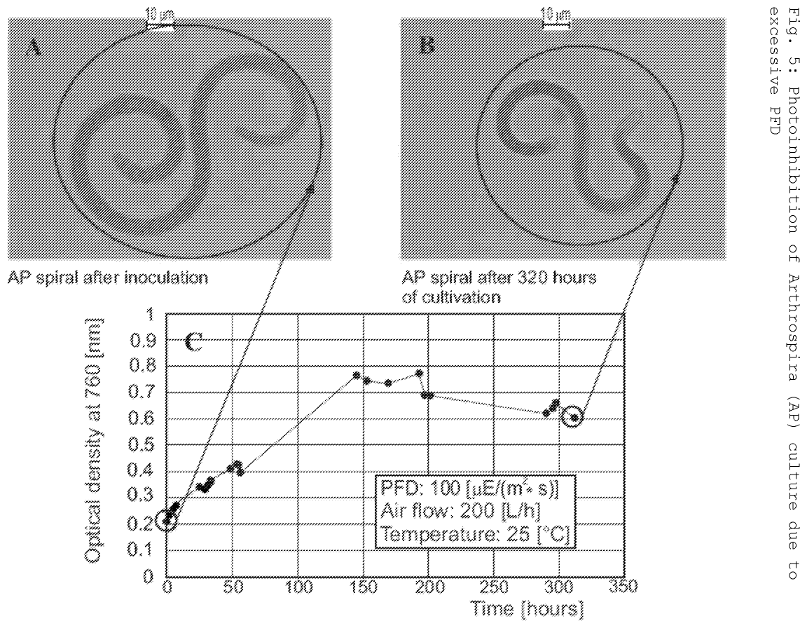

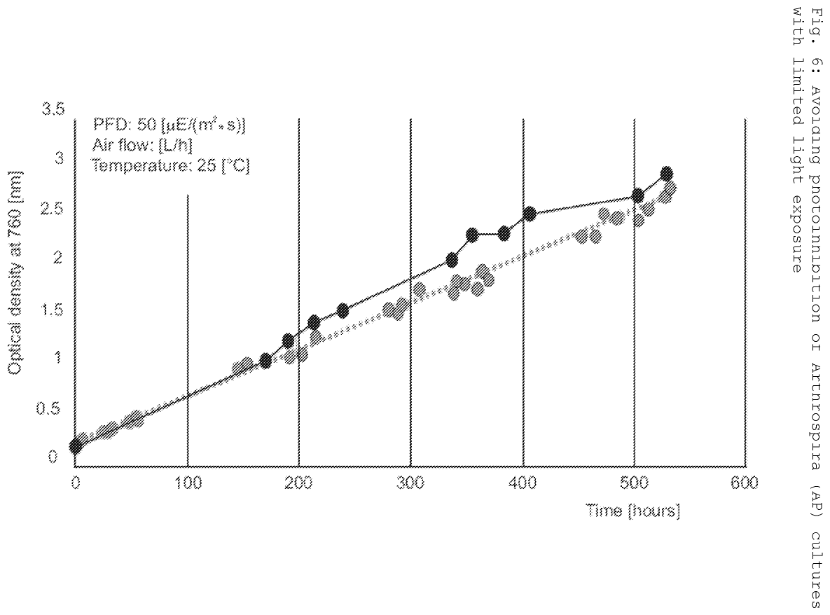

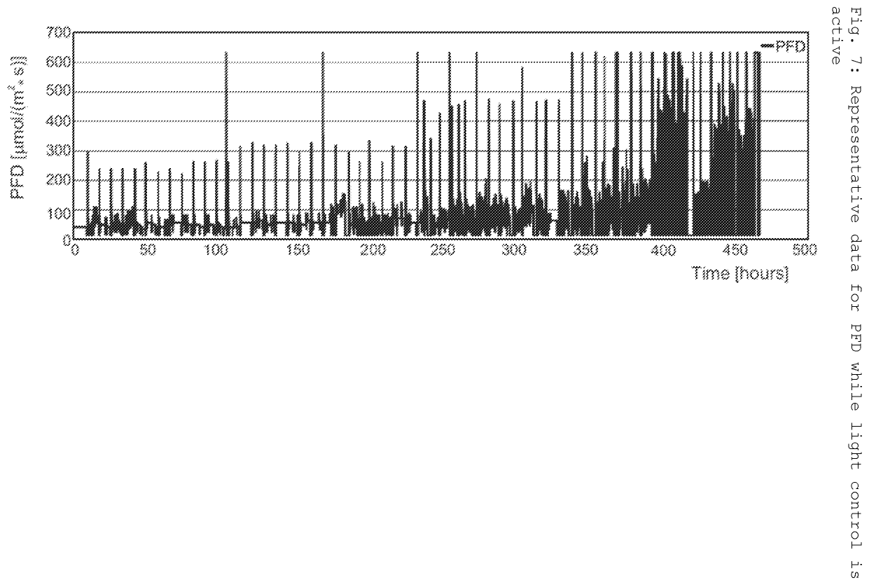

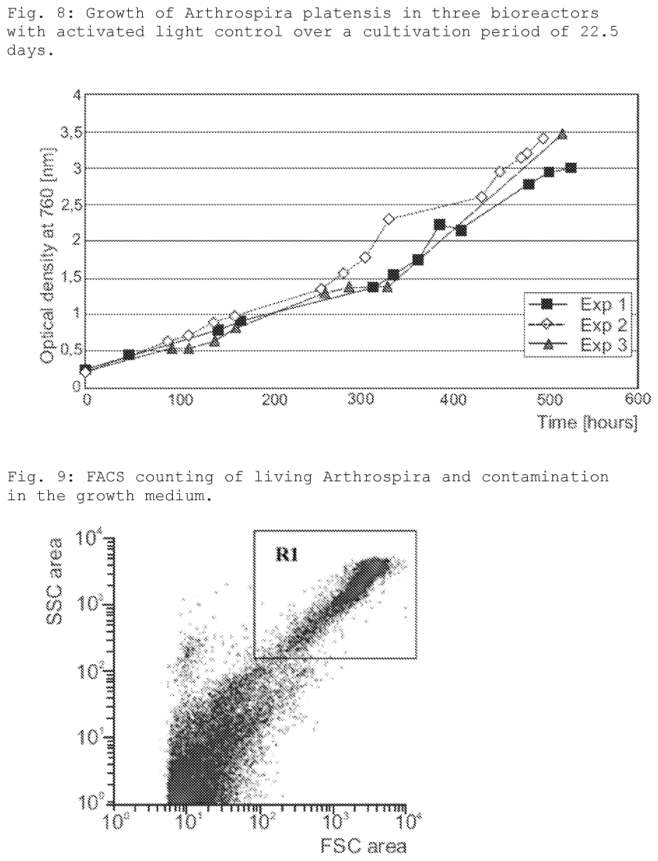

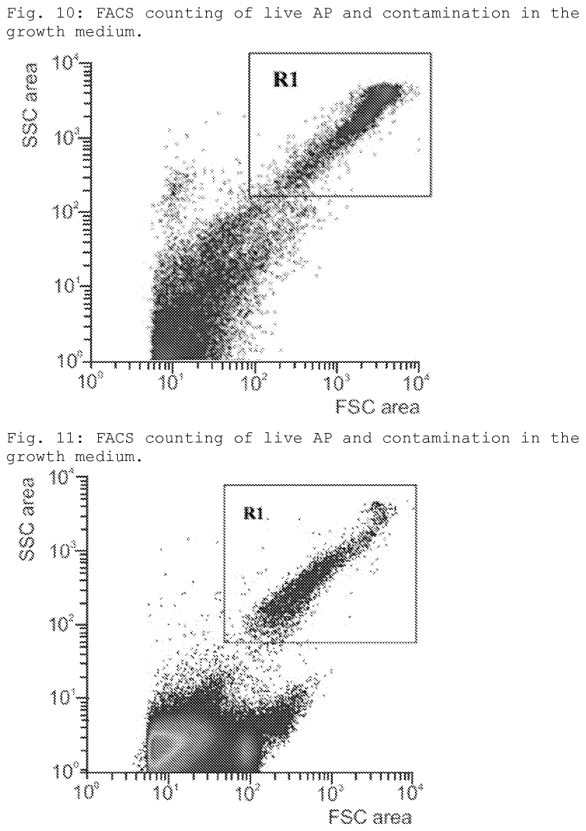

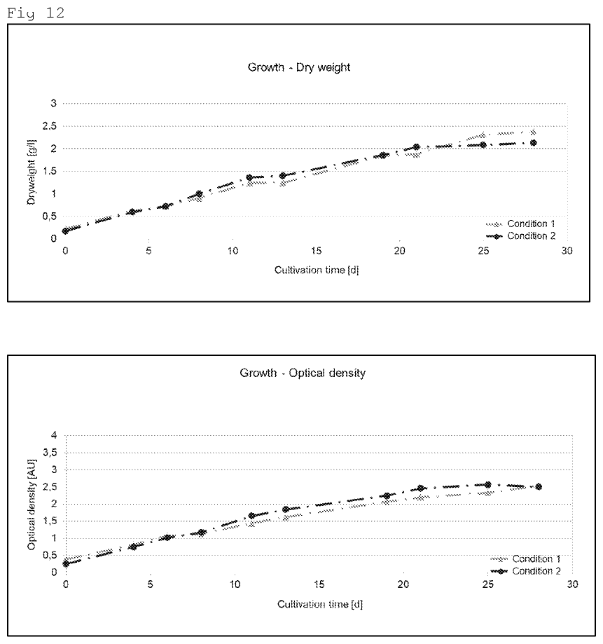

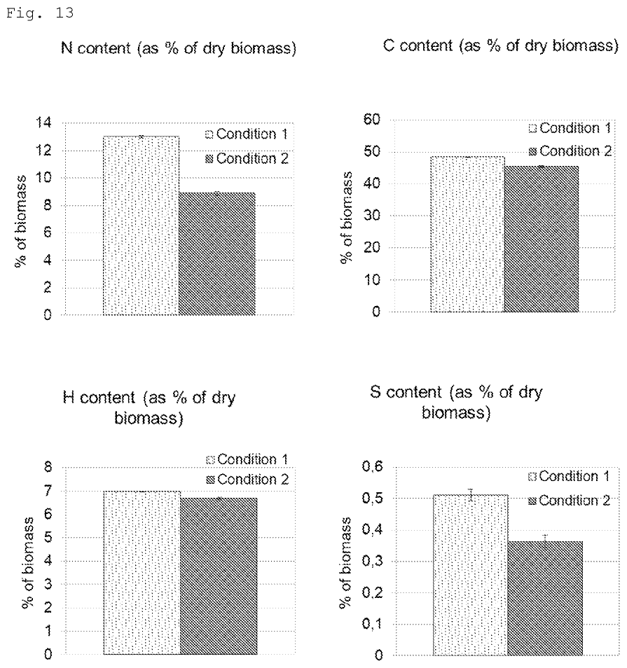

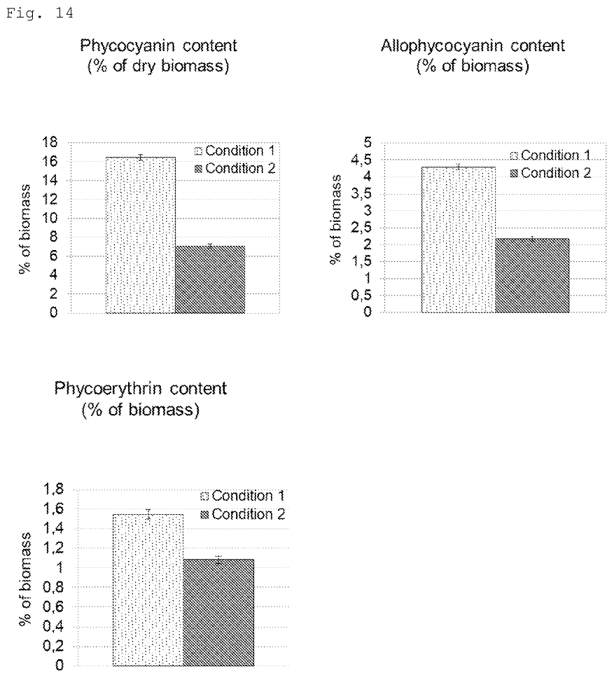

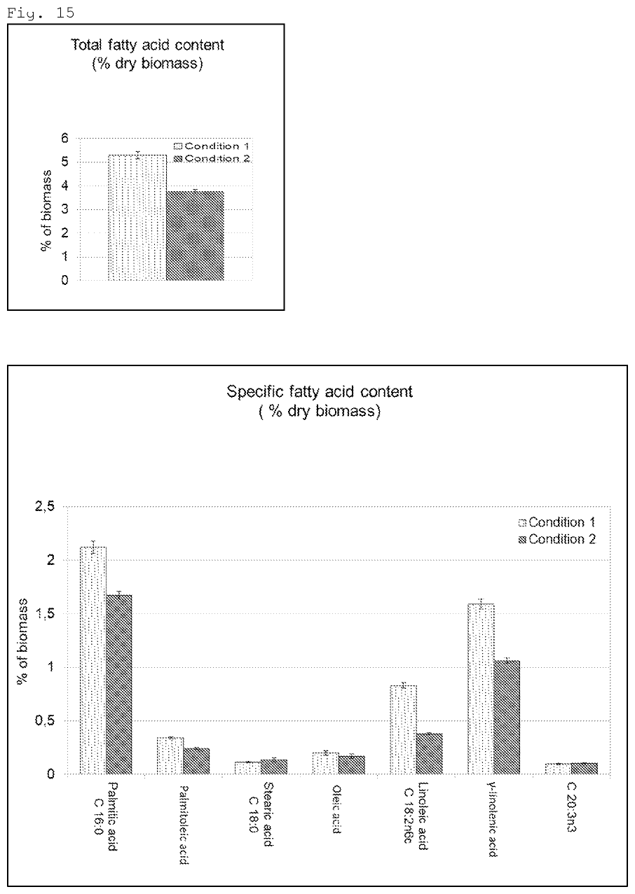

A) and C) in FIG. 1 show images of Arthrospira with a laser scanning microscope (ZEISS LSM 800), where Arthrospira shows the expected normal morphology of spiral microalgae with internal granular structures. These are due to the photosynthetic pigments located in intracellular membrane stacks. In B) and D) an Arthrospira spiral is shown, which originated from a culture in which no hydrogen carbonate was added and the microalgae were additionally exposed to a high PFD for 1 h. The granular structures largely disappear, which can already be seen in transmission mode (A and B) and even better with AERYSCAN (C and D).

FIG. 2:

Arthrospira cultures of low density that grow without additional hydrogen carbonate and are exposed to a high PFD can even die off completely within a short time.

FIG. 3:

Representation of the course of a PFD adjustment with control unit over time in correlation to the pH value.

FIG. 4:

Self-optimizing determination of the pH setpoint.

FIG. 5:

Photoinhibition of Arthrospira (AP) culture by too high PFD according to example 1.

FIG. 6:

Avoidance of photoinhibition of Arthrospira (AP) cultures with limited light exposure

FIG. 7:

Representative data on the PFD while light control is active.

FIG. 8:

Growth of Arthrospira platensis in three bioreactors with activated light control over a cultivation period of 22.5 days.

FIG. 9: FACS counting of living Arthrospira and of contaminants in the growth medium. For Arthrospira production, the method according to the invention was used with atmospheric CO₂ as the carbon source and the CO₂ supply was carried out in an open, exposed bioreactor (Open Pond System, 500 liter cultivation volume) with air gassing. Before the measurement, the culture medium was filtered to remove the large Arthrospira particles. The permeate was then measured using flow cytometry. In the example, 56,504 particles were counted, of which 8979 particles were located in the R1 field, in which Arthrospira (AP) was previously clearly identified on the basis of fluorescence (FL4). The contaminations have significantly less fluorescence and can be clearly identified by FSC and SSC.

FIG. 10:

FACS counting of live Arthrospira (AP) and contamination in the growth medium. For Arthrospira (AP) production, the standard procedure with Zarrouk complete medium including an added carbon source in the form of HCO₃⁻ was used. In the example, 737,844 particles are counted in the permeate, of which 4953 particles are located in the field R1, in which AP could be clearly identified on the basis of fluorescence (FL4). The contaminations have significantly less fluorescence and can be clearly identified by FSC and SSC.

FIG. 11:

For Arthrospira (AP), the standard method with Zarrouk complete medium including an added carbon source in the form of HCO₃⁻ was used. In the example, 737,844 particles are counted in the permeate, of which 4953 particles are located in the field R1, in which AP could be clearly identified due to the fluorescence (FL4). The contaminations have significantly less fluorescence and can be clearly identified by FSC and SSC.

FIG. 12:

Arthrospira cells were cultivated for 4 weeks according to Condition 1 (only atmospheric CO₂ as carbon source) or Condition 2 (NaHCO₃ weighed in medium plus an additional 2% technical CO₂ in the air stream served as carbon source). The increase in biomass (dry weight) and optical density over time is shown.

FIG. 13:

Arthrospira cells were cultured for 4 weeks according to condition 1 (only atmospheric CO₂ as carbon source) or condition 2 (NaHCO₃ weighed in medium served as carbon source plus additional 2% technical CO2 in the air stream). An elemental analysis was carried out for nitrogen (N), carbon (C), hydrogen (H) and sulphur (S). The values at the end of the test period are shown as a percentage of the dry biomass.

FIG. 14:

Arthrospira cells were cultured for 4 weeks according to condition 1 (only atmospheric CO2 as carbon source) or condition 2 (NaHCO₃ weighed in medium served as carbon source plus additional 2% technical CO2 in the air stream). The amount of phycobiliproteins was determined. The values at the end of the test period are shown as a percentage of the dry biomass.

FIG. 15:

Arthrospira cells were cultivated for 4 weeks according to Condition 1 (only atmospheric CO2 as carbon source) or Condition 2 (NaHCO₃ weighed in medium plus an additional 2% technical CO2 in the air stream served as carbon source. The total fatty acids and the proportion of specific fatty acids were determined at the end of the test period (in % of the dry biomass).

### EXAMPLES

### Example 1

An Arthrospira culture is cultivated in a Zarrouk medium, but without the addition of hydrogen carbonate for 13 days, using a PFD of 100 μE/m2*sec and an air flow of 200 L/h. Under the selected cultivation conditions, photoinhibition of the culture occurred after approx. 150 hours, so that no further increase in optical density was observed (FIG. 5). The experiment was terminated after 200 hours.

### Example 2

An Arthrospira culture is cultivated in hydrogen carbonate-free Zarrouk medium for 13 days, using a PFD of 50 μE/m2*sec and an air flow of 200 L/h. Under the selected cultivation conditions, there is no recognizable photoinhibition of the culture, so that it continues to grow linearly (FIG. 6).

At the end of the experiment, an optical density of just under 3 was achieved.

However, the experiments shown above show the cultivation process with a fixed PFD.

FIG. 7 shows the regulation of the PFD based on the pH value of the AP culture and FIG. 8 the resulting optical density of the cultures in three independent test runs.

### Example 3

a.) Contamination During Growth of Arthrospira (AP) Under Fumigation with Ambient Air, in the Air Stream (without Fossil C Source)

The measurement of contamination by particles other than Arthrospira (e.g. heterotrophic bacteria) was carried out using a flow cytometer (FACS) in the culture medium (200 μl). The method is known to the skilled person. First, the large Arthrospira microfilaments were largely filtered off in order to avoid blockages during flow cytometry. First, the area of living Arthrospira (AP) remaining in the permeate in the sample was determined by fluorescence (FL4 channel of the FACS device: excitation: 561 nm+488 nm, emission: 615 nm, ±25 nm) (not shown). On this basis, an area R1 was defined, which contains the arthrospira cells present in the permeate, which are visible due to their fluorescence. The intrinsic fluorescence of the Arthrospira (phycoerythrin) is used here; the contaminants do not fluoresce or fluoresce considerably less and can therefore be distinguished from Arthrospira (see appendix). Subsequently, both the previously defined area R1 and the other areas were examined using the FSC (Forward Scatter=size) and SSC (Side Scatter=granularity) settings of the flow cytometer (FIG. 9). The “total events” of particles could be measured and these could be set in relation to the Arthrospira particles still in the permeate.

Outside the R1 area is the non-fluorescent or low-fluorescent area, which consists of debris and contamination, so that quantification can be carried out using FACS.

A total of 56,504 events were counted in the 200 μl measurement medium, from which 8,979 live APs must be subtracted. This results in a total number of 47,522 contaminations.

b.) Contamination During the Growth of spirulina with Zarrouk Complete Medium

Here, under identical measurement conditions, 4,953 live Arthrospira are found in the permeate with a total number of 737,844 measurement events. This results in a number of 732,891 contaminations.

This clearly shows that the process according to the invention results in considerably less contamination in the air-fumigated pond (15.4 times less contamination) than in the bioreactor with Zarrouk complete medium (according to example 4).

### Example 4

1) Composition of the Zarrouk culture medium according to the state of the art per liter of water:
   18 g NaHCO3
   2.5 g NaNO₃
   1 g K₂ SO₄
   1 g NaCl
   0.2 g MgSO₄-7H O₂
   0.04 g CaCl₂
   0.01 g FeSO₄-7H O₂
   0.08 g Na₂ EDTA
   and 1 ml of a trace element solution.
   The trace metal solution consists of (per liter): 2.86 g H₃ BO₃, 1.81 g MnCl₄-4H₂ O, 0.222 g ZnSO₄-4H₂ O, 0.0177 g Na₂ MoO₄, 0.079 g CuSO₄-5H O.₂
2) Composition of a culture medium according to the invention per liter of water containing no carbon source:
   0.00 g NaHCO₃
   0.5 g K₂ HPO₄
   2.5 g NaNO₃
   1 g K₂ SO₄
   1 g NaCl
   0.2 g MgSO₄-7H O₂
   0.04 g CaCl₂
   0.01 g FeSO₄-7H O₂
   0.08 g Na₂ EDTA
   and 1 ml of a trace element solution.
   The trace metal solution consists of (per liter): 2.86 g H₃BO₃, 1.81 g MnCl₄ 4H₂O, 0.222 g ZnSO₄ 4H₂O, 0.0177 g Na₂MoO₄, 0.079 g CuSO₄ 5H₂O.

### Example 5

An Arthrospira culture was cultivated in Zarrouk medium without the addition of NaHCO₃ (supra) for 4 weeks, using a photon flux density (PFD) of 30 μE/m2*sec and an air flow of 500 L/h (Condition 1). The air flow contains the atmospheric CO₂ concentration present at this time (approx. 0.04%). Under the selected cultivation conditions, there is no recognizable photoinhibition of the culture, so that it continues to grow linearly.

In comparison, an Arthrospira culture was cultivated in Zarrouk medium including the addition of NaHCO₃(Zarrouk complete medium) for 4 weeks, using a PFD of 30 μE/m2*sec. The culture was adjusted with an air flow of 25 L/h, in which an additional CO₂ flow of 2% was added (Condition 2). Under the selected cultivation conditions, there is no recognizable photoinhibition of the culture, so that it continues to grow linearly.

The following measurements were carried out with Arthrospira cells propagated under these two culture conditions:

Firstly, a growth curve was drawn up over time based on the optical density (OD) and dry biomass (dry weight). Under both culture conditions, an optical density of approx. 2.5 was achieved, which corresponds approximately to a biomass concentration (dry weight) of 2-2.5 g/l. There are thus no recognizable differences in the growth curves of the Arthrospira cultures of Condition 1 and Condition 2 (see FIG. 12).

The content of nitrogen (N), carbon (C), hydrogen (H) and sulphur (S) was determined using an elemental analysis (Euro Vector elemental analyzer). The elemental analysis showed that the nitrogen and sulphur content of the Arthrospira cells cultivated under Condition 1 was significantly higher than that of the cells harvested under Condition 2. A higher nitrogen content of the biomass indicates a higher amount of protein according to the Kjeldahl method known to the skilled person. With a conversion factor of 6.25 for Arthrospira platensis (Piorreck, M., Baasch, K.-H., Pohl, P., 1984. Biomass production, total protein, chlorophylls, lipids and fatty acids of freshwater green and blue-green algae under different nitrogen regimes. Phytochemistry 23, 207-216. https://doi.org/10.1016/S0031-9422(00)80304-0) the following differences in protein levels can be determined: 4 weeks of cultivation in Condition 1 leads to an N content of 13.0145 wt. % and thus to a protein content of 81.3 wt. %. In Condition 2, an N content of 8.90625% by weight and a protein content of 55.7% by weight were obtained. The carbon and hydrogen content at the end of the test period is also higher in the Arthrospira cells propagated under Condition 1 than under Condition 2. FIG. 13 shows the differences in the elements N, C, H and S for Condition 1 and Condition 2.

Using the photometric method known to the skilled person (Bennett A. & Bogorad L. J Cell Biol. 1973 Aug. 1; 58(2): 419-435. doi: 10.1083/jcb.58.2.419) was used to determine the content of the phycobiliproteins phycocyanin, allophycocyanin and phycoerythrin. It can be seen that the content of phycobiliproteins after 4 weeks is generally significantly higher under Condition 1 than under Condition 2; the content of phycocyanin is even twice as high under Condition 1 as under Condition 2 (cf. FIG. 14).

The method of Bligh and Dyer, which is well known to experts (Bligh, E. G. & Deyer, W. J. Can. J. Biochem. Physiol. Vol. 37: 911-917, 1959), the fatty acids present in the cells in lipid form were converted into fatty acid methyl esters (FAME). These are analyzed with a gas chromatograph (Agilent 7820A, GC column SP2560 from Supelco, using biscyanopropyl polysiloxane). It can be seen that the absolute content of fatty acids and also the content of some specific fatty acids such as palmitic acid is higher in the Arthrospira cells grown under Condition 1 than in Condition 2 (see FIG. 15).

1. A process for sequestering carbon from the air, wherein air is supplied to autotrophic microorganisms in at least one bioreactor, and carbon dioxide from the air is a sole carbon source for the autotrophic microorganisms.

2. The process for sequestering carbon according to claim 1, wherein the at least one bioreactor is a photobioreactor or open pond bioreactor.

3. The process for sequestering carbon from the air according to claim 1, wherein the autotrophic microorganisms are photoautotrophic microorganisms or chemoautotrophic microorganisms.

4. The process for sequestering carbon from the air according to claim 1, wherein the autotrophic microorganism is Arthrospira.

5. The process for sequestering carbon from the air according to claim 1, wherein the culture medium for the autotrophic microorganisms does not contain a carbon source.

6. The process for sequestering carbon from the air according to claim 1, wherein the culture medium for the autotrophic microorganisms does not contain a carbon source and is selected from the group consisting of:
0.5 g K₂HPO₄,
2.5 g NaNO₃,
1 g K₂SO₄,
1 g NaCl,
0.2 g MgSO₄ 7H₂O,
0.04 g CaCl₂,
0.01 g FeSO₄ 7H₂O,
0.08 g Na₂EDTA, and
1 ml of a trace element solution
per 1 liter of water.

7. The process for sequestering carbon from the air according to claim 1, wherein a pH of the culture medium is 7 to 11, and wherein the pH of the culture medium is adjusted via photon flux density (PFD) and/or with an air stream and/or with additional carbon dioxide in the air stream.

8. The process for sequestering carbon from the air according to claim 1, wherein a pH of the culture medium represents a setpoint value between 7 to 11, and deviations from the setpoint value are controlled via photon flux density (PFD) and/or with an air stream and/or with additional carbon dioxide in the air stream.

9. The process for sequestering carbon from the air according to claim 7, wherein the photon flux density (PFD) is greater than 0-10,000 μE/m²*sec*m² and/or the air is supplied to the autotrophic microorganisms in the at least one bioreactor with an air flow of 50-50,000 L/h*m².

10. The process for sequestering carbon from the air according to claim 7, wherein a proportion of carbon dioxide in the air stream is from 0.1-5% by volume.

11. The process for sequestering carbon from the air according to claim 1, wherein a temperature in the at least one bioreactor is between 2° and 45° C.

12. A biomass containing autotrophic microorganisms, obtainable by a process according to claim 1, wherein a nitrogen content in the biomass is at least 10% by weight, or a protein content is more than 70% by weight, in particular more than 75% by weight.

13. The process for sequestering carbon according to claim 3, wherein the autotrophic microorganisms are archaebacteria, algae, microalgae, and/or cyanobacteria.

14. The process for sequestering carbon according to claim 3, wherein the autotrophic microorganisms are algae of the genera Chlorella, Scenedesmus, Arthrospira, Nannochloropsis, Nostoc, and/or Chlorococcus.

15. The process for sequestering carbon according to claim 4, wherein the autotrophic microorganism is A. platensis, A. maxima, and/or A. fusiformis.

16. The biomass according to claim 12, wherein the autotrophic microorganisms are algae of the genus Arthrospira.

17. The biomass according to claim 12, wherein the autotrophic microorganism is A. platensis, A. maxima, and/or A. fusiformis.
